(19) United States
(12) Patent Application Publication
(10) Pub. No.: US 2025/0352232 A1
(43) Pub. Date: Nov. 20, 2025

(54) MEDICAL DEVICE

(71) Applicant: OLYMPUS MEDICAL SYSTEMS CORP., Tokyo (JP)

(72) Inventors: Mitsuaki HASEGAWA, Hino-shi (JP); Tatsuya SUZUKI, Hachioji-shi (JP); Kensuke Uesaka, Hino-shi (JP); YuYa HIDAKA, Hino-shi (JP); Kazuhiro MORISAKI, Yokohama-shi (JP)

(21) Appl. No.: 19/071,519

(22) Filed: Mar. 5, 2025

Related U.S. Application Data
(60) Provisional application No. 63/649,465, filed on May 20, 2024.

(51) Int. Cl. A61B 17/29; A61B 17/00; A61B 17/10

(52) U.S. Cl. CPC A61B 17/2909; A61B 17/105; A61B 2017/00438; A61B 2017/2912; A61B 2017/2932

(57) ABSTRACT

A medical device includes: a sheath extending in a longitudinal direction; a treatment portion disposed at a distal end of the sheath; a wire inserted into the sheath and to which the treatment portion is fixed; a handle disposed at a proximal end of the sheath and to which the wire is fixed; and a transmission member disposed on an outer periphery of the sheath and extending from the handle to the distal end, wherein the treatment portion, the wire, the handle, and the transmission member are rotatable in a circumferential direction with respect to the longitudinal direction relative to the sheath.

### CROSS-REFERENCE TO RELATED APPLICATIONS

This application claims priority to U.S. Provisional Patent Application No. 63/649,465, filed May 20, 2024, the contents of which are incorporated herein by reference.

### TECHNICAL FIELD

The present disclosure relates to a medical device equipped with an endoscopic treatment portion.

### BACKGROUND ART

In endoscopic treatment, treatment portions such as clip units and forceps are used. The treatment portion is introduced to the treatment position by an applicator (introduction device) that can be inserted through the channel of the endoscope. The applicator includes, for example, a wire connected to the treatment portion, a handle for operating the treatment portion via the wire, and a sheath through which the wire is inserted.

When performing treatment using the treatment portion, it is necessary to rotate the treatment portion appropriately. For example, U.S. Patent Application Publication No. 2017/0224341 (which is referred to as Patent Document 1) describes a medical device having a configuration in which the treatment tool is rotated by rotating the sheath. When performing treatment using the treatment portion, the sheath is inserted into the channel of the endoscope, so the treatment tool can be rotated by rotating the part of the sheath exposed from the channel of the endoscope.

In the medical device described in Patent Document 1, when the treatment portion is rotated by rotating the sheath, the sheath comes into contact with the inner wall of the endoscope channel, generating friction, which makes it difficult for the rotation of the sheath to be transmitted to the treatment portion, making it difficult for the treatment portion to rotate.

### SUMMARY

The present disclosure provides a medical device that makes it easy to rotate the treatment portion when the sheath is inserted into the endoscope channel.

The medical device according to a first aspect of the present disclosure includes: a sheath extending in a longitudinal direction; a treatment portion disposed at a distal end of the sheath; a wire inserted into the sheath and to which the treatment portion is fixed; a handle disposed at a proximal end of the sheath and to which the wire is fixed; and a transmission member disposed on an outer periphery of the sheath and extending from the handle to the distal end, wherein the treatment portion, the wire, the handle, and the transmission member are rotatable in a circumferential direction with respect to the longitudinal direction relative to the sheath.

The medical device of the present disclosure makes it easy to rotate the treatment portion when the sheath is inserted into the channel of an endoscope, and treatment using the treatment portion can be smoothly performed.

### BRIEF DESCRIPTION OF DRAWINGS

FIG. 1 is a side view showing a clip device according to a first embodiment.

FIG. 2 is a perspective view showing the clip introduction device.

FIG. 3 is a side view showing an operation portion, with a partial cross section.

FIG. 4 is a perspective view showing an outer sheath, showing the configuration of the outer sheath.

FIG. 5 is a perspective view showing a clip unit.

FIG. 6 is a plan view showing a cartridge.

FIG. 7 is a view showing the clip unit connected to the clip introduction device.

FIG. 8 is a view showing the clip unit connected to the clip introduction device.

FIG. 9 is a view showing the clip unit being pulled out of the cartridge.

FIG. 10 is a view showing the state in which the sheath of the clip device has been inserted into the treatment tool channel of an endoscope.

FIG. 11 is a perspective view showing the endoscope system in use.

FIG. 12 is a view showing the state in which the sheath of the clip device has been inserted into the treatment tool channel of an endoscope.

FIG. 13 is a perspective view showing the state in which the endoscope system is in use.

FIG. 14 is a cross-sectional view of the operation portion according to a first modification, showing a cross-section along the longitudinal direction.

FIG. 15 is an enlarged view of the range indicated by dashed line D13 of the same operation portion shown in FIG. 14.

FIG. 16 is a side view of the operation portion according to a second modification, showing a partial cross-section.

FIG. 17 is a cross-sectional view of the same operation portion, showing a cross-section along the longitudinal direction.

FIG. 18 is a cross-sectional view along line F14-F14 of the same operation portion shown in FIG. 16.

FIG. 19 is an enlarged view of the range indicated by dashed line D17 of the same operation portion shown in FIG. 18.

FIG. 20 is a side view of the operation portion main body, showing a cross-section of the slider.

FIG. 21 is a cross-sectional view along line F19-F19 of the same operation portion shown in FIG. 16.

FIG. 22 is an enlarged view of the range indicated by dashed line D20 of the same operation portion shown in FIG. 21.

FIG. 23 is a cross-sectional view of the operation portion pertaining to a third modification, showing a cross section along the longitudinal direction.

FIG. 24 is a side view showing an outer sheath pertaining to a fourth modification.

FIG. 25 is a side view showing an outer sheath configured to be extendable and contractible.

FIG. 26 is a side view showing a transmission member configured to be extendable and contractible in the longitudinal direction.

FIG. 27 is a side view showing a clip device pertaining to a second embodiment.

FIG. 28 is a view showing the sheath of the clip device inserted into a treatment tool channel of an endoscope.

FIG. 29 is a side view showing a clip device pertaining to a fifth modification.

### DESCRIPTION OF EMBODIMENTS

An embodiment of the medical device according to the present disclosure will be described with reference to the drawings. In the following description, components having the same or similar functions will be given the same reference numerals. Duplicated descriptions of those components may be omitted. In this embodiment, a clip device will be described as an example of a medical device.

### First Embodiment

### [Clip Device 300]

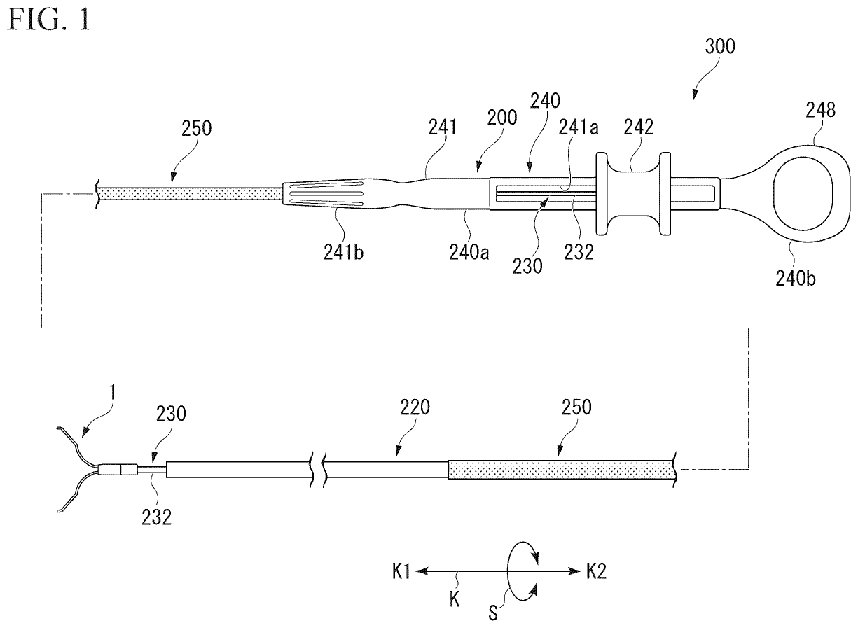

A clip device 300 according to a first embodiment of the present disclosure will be described with reference to FIGS. 1 to 26.

FIG. 1 is a side view showing the clip device 300 according to the first embodiment.

The clip device 300 includes a clip introduction device (applicator) 200 and a clip unit 1. The clip unit 1 is loaded into the clip introduction device 200.

### [Clip Introduction Device 200]

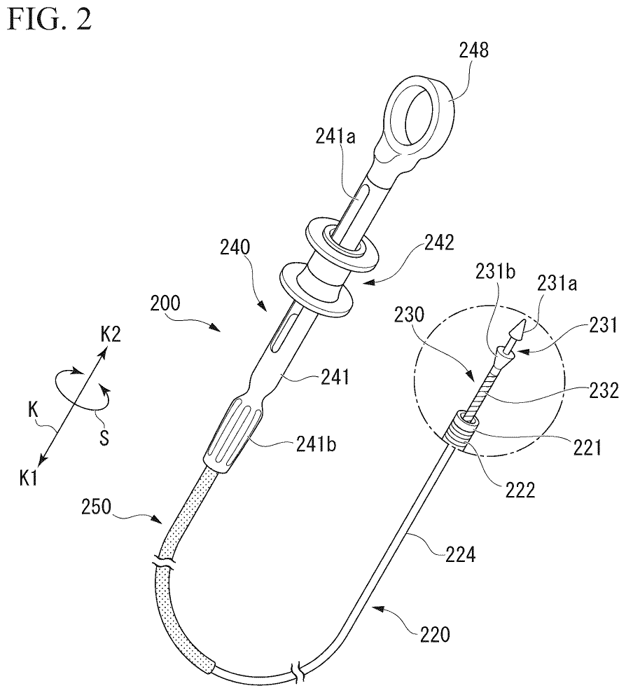

FIG. 2 is a perspective view showing the clip introduction device 200.

As shown in FIG. 2, the clip introduction device 200 includes a sheath 220, an operating wire 230, an operation portion (handle) 240, and an outer sheath (transmission member) 250.

In the clip introduction device 200, the direction in which the sheath 220 extends is the longitudinal direction K. Within the longitudinal direction K, the distal end side is designated as K1, and the proximal end side is designated as K2. The circumferential direction with respect to the longitudinal direction K is designated as the circumferential direction S.

As shown in FIG. 2, the sheath 220 comprises a distal tip 221, a distal coil 222, and a proximal coil 224, and is formed into an elongated tube shape overall. The sheath 220 is a member extending in the longitudinal direction K. The distal coil 222 is disposed on the distal end side of the sheath 220. The distal tip 221 is disposed at the distal end of the distal coil 222.

The sheath 220 is inserted into the treatment tool channel (endoscope channel) 430 of the endoscope 400 described later, and is used in combination with the endoscope. Therefore, the sheath 220 is formed to be sufficiently longer than the length M6 of the treatment tool channel 430. The sheath 220 is flexible and curves to match the treatment tool channel 430.

The sheath 220 is provided to be rotatable relative to the operation wire 230 in the circumferential direction S. The sheath 220 is provided to be rotatable relative to the operation portion 240 in the circumferential direction S. The sheath 220 is provided to be rotatable relative to the outer sheath 250 in the circumferential direction S. The sheath 220 is provided to be rotatable relative to the clip unit 1 in the circumferential direction S.

The operation wire (power transmission unit) 230 is inserted into the sheath 220 as shown in FIG. 2. The operating wire 230 includes an arrowhead hook (connection portion) 231 connected to the clip unit 1, and a wire 232 for operating the arrowhead hook 231.

As shown in FIG. 2, the arrowhead hook 231 includes an engaging portion 231a having a generally conical shape that engages with the clip unit 1, and a wire connecting portion 231b provided at the proximal end of the engaging portion 231a. The arrowhead hook 231 is formed of a metal material such as stainless steel.

The wire 232 is inserted through the sheath 220 so as to be freely movable forward and backward. The distal end of the wire 232 is fixed to the proximal end of the wire connecting portion 231b by welding, for example.

The operating wire 230 is provided so as to be rotatable relative to the sheath 220 in the circumferential direction S. The operating wire 230 is fixed to the operating portion 240, and rotates in the circumferential direction S in conjunction with the operating portion 240. The operating wire 230 is provided with respect to the outer sheath 250 so as not to rotate relative to it in the circumferential direction S. The operating wire 230 is connected to the clip unit 1 and rotates in the circumferential direction S in conjunction with the clip unit 1.

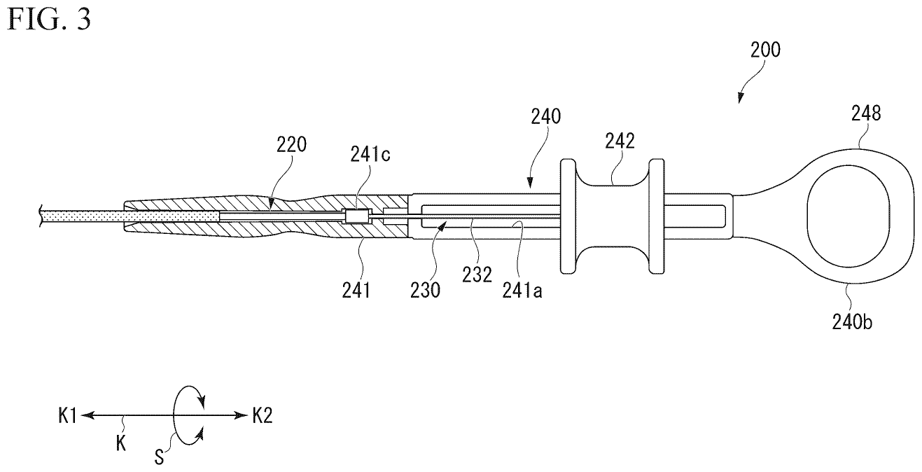

FIG. 3 is a side view of the operation portion 240, with a partial cross section.

As shown in FIG. 2, the operation portion 240 is disposed on the proximal end side K2 of the sheath 220. As shown in FIG. 3, the operation portion 240 includes an operation portion main body (handle main body) 241, a slider 242, and a thumb ring 248. The operation portion main body 241, the slider 242, and the thumb ring 248 are injection molded from, for example, a resin material. The operation portion main body 241 includes a slit portion 241a and a rotating grip 241b on the distal end side. The slit portion 241a supports the slider 242 so that it can advance and retract.

The slider 242 is attached to the operation portion main body 241 so as to be movable in the longitudinal direction K, and the proximal end of the wire 232 is attached to the slider 242. The slider 242 is supported by the operation portion main body 241. When the slider 242 advances and retracts along the operation portion main body 241, the wire 232 advances and retracts relative to the sheath 220, and the arrowhead hook 231 advances and retracts.

The thumb ring 248 is attached to the proximal end of the operation portion main body 241 so as to be rotatable in the circumferential direction S relative to the operation portion main body 241. The thumb ring 248 rotates in the circumferential direction S relative to the operation portion main body 241, with the longitudinal direction K as the axis of rotation.

As shown in FIG. 3, a retainer 241c is provided inside the operation portion main body 241. The retainer 241c is formed in a cylindrical shape extending in the longitudinal direction K. The retainer 241c is provided rotatably in the circumferential direction S relative to the operation portion main body 241. The retainer 241c rotates in the circumferential direction S relative to the operation portion main body 241 with the longitudinal direction K as the axis of rotation. The range in which the retainer 241c can move in the longitudinal direction K is limited. The range in which the retainer 241c can move in the longitudinal direction K is shorter than the length of the retainer 241c in the longitudinal direction K. The retainer 241c does not have to be movable in the longitudinal direction K. The sheath 220 is fixed to the distal end of the retainer 241c. The sheath 220 and the retainer 241c move together.

The operation portion 240 has a first part 240a connected to the outer sheath 250 and a second part 240b connected to the first part 240a so as to be rotatable relative to the first part 240a in the circumferential direction S. The first part 240a has an operation portion main body 241 and a slider 242. The second part 240b has a thumb ring 248.

The operation portion 240 is provided so as to be rotatable relative to the sheath 220 in the circumferential direction S. The operation portion 240 is fixed to the operating wire 230, and rotates in the circumferential direction S in conjunction with the operating wire 230. The operation portion 240 is fixed to the outer sheath 250, and rotates in the circumferential direction S in conjunction with the outer sheath 250.

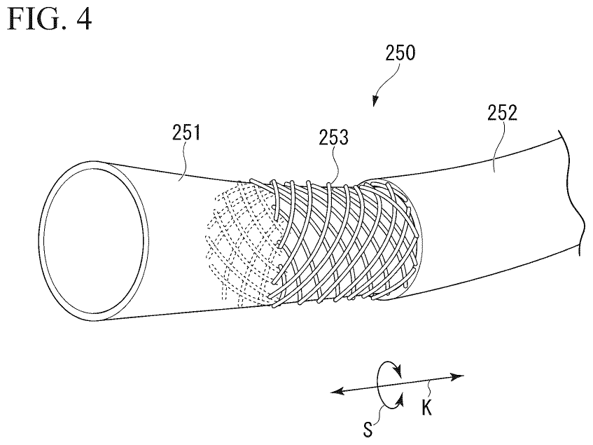

FIG. 4 is a perspective view showing the outer sheath 250, and shows the configuration of the outer sheath 250.

The outer sheath 250 is disposed on the outer peripheral side of the sheath 220, as shown in FIG. 1. Specifically, the sheath 220 is inserted into the outer sheath 250. The outer sheath 250 is formed in a cylindrical shape through which the sheath 220 is inserted, as shown in FIG. 3. The outer sheath 250 extends from the distal end of the operation portion main body 241 to the distal end side K1 in the longitudinal direction K, as shown in FIG. 3. The outer sheath 250 is fixed to the operation portion main body 241. The outer sheath 250 has a higher flexibility than the operation portion 240 and can be bent along the sheath 220. The length M5 of the outer sheath 250 in the longitudinal direction K is longer than the length M4 of the operation portion 240 in the longitudinal direction K. The length M5 of the outer sheath 250 in the longitudinal direction K is shorter than the length M9 of the sheath 220 in the longitudinal direction K.

The outer sheath 250 has an inner layer portion 251, an outer layer portion 252, and a middle layer portion 253 disposed radially between the inner layer portion 251 and the outer layer portion 252, as shown in FIG. 4. The outer sheath 250 is, for example, a braid-in-tube. The inner layer 251 is formed containing resin. The outer layer 252 is formed containing resin. The middle layer 253 is a member having a higher hardness than the inner layer 251. The middle layer 253 is a member having a higher hardness than the outer layer 252. The outer layer 252 may be a member having a higher hardness than the inner layer 251 and the middle layer 253. The middle layer 253 is formed containing metal wire.

The outer sheath 250 is provided so as to be rotatable relative to the sheath 220 in the circumferential direction S. The outer sheath 250 is provided so as not to be rotatable relative to the operating wire 230 in the circumferential direction S. The outer sheath 250 is fixed to the operation portion 240 and rotates in the circumferential direction S in conjunction with the operation portion 240. The clip unit 1, the operating wire 230, the operating portion 240, and the outer sheath 250 are rotatable relative to the sheath 220 in the circumferential direction S.

### [Clip Unit 1]

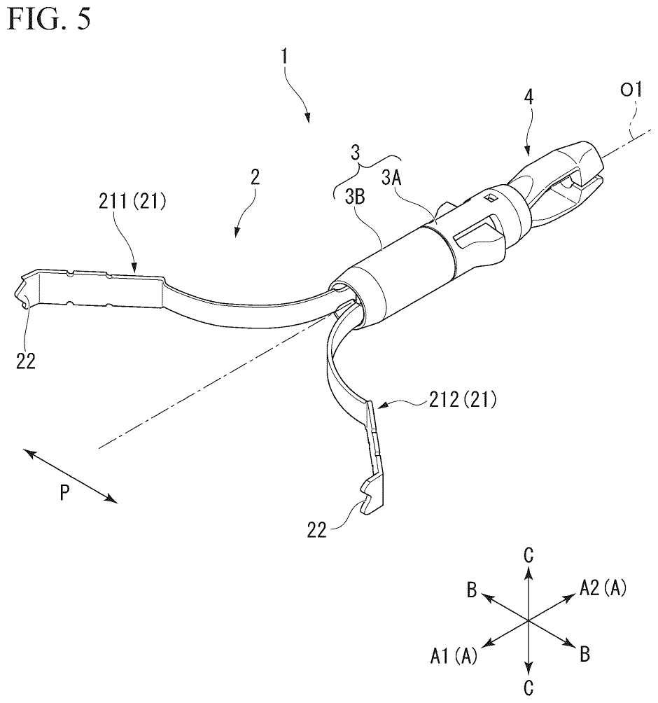

FIG. 5 is a perspective view showing the clip unit 1.

As shown in FIG. 1, the clip unit 1 is disposed on the distal end side K1 of the sheath 220. As shown in FIG. 5, the clip unit 1 includes a clip 2, a pressing member 3, and a connecting member 4.

The clip unit 1 is a “treatment portion” that is inserted into the treatment tool channel 430 of the endoscope 400 and used to treat an affected area.

The clip unit 1 and the sheath 220 are not fixed. Furthermore, the clip unit 1 can rotate relative to the sheath 220 in the circumferential direction S.

In the following explanation, the clip 2 side of the clip unit 1 in the longitudinal direction A is the distal end side (distal side) A1 of the clip unit 1, and the side of the connecting member 4 is the proximal end side (proximal side) A2 of the clip unit 1. The direction perpendicular to the longitudinal direction A is referred to as the “second direction B” or the “left-right direction B”. The direction perpendicular to the longitudinal direction A and the second direction B is referred to as the “first direction C” or the “up-down direction C”.

The clip (clip arm) 2 is formed by bending a metal plate at the center. As shown in FIG. 5, the clip 2 has a pair of arms 21 that can be opened and closed. The proximal end side A2 of the clip 2 is inserted into the internal space of the pressing member 3.

The pair of arms 21 has a first arm 211 and a second arm 212 as shown in FIG. 5. The first arm 211 and the second arm 212 are disposed on both sides of the central axis O1 in the longitudinal direction A of the clip unit 1. The clip 2 may have three or more arms.

The first arm 211 and the second arm 212 have tissue grasping portions 22 formed at the ends of the distal end side A1 as shown in FIG. 5. The tissue grasping portion 22 is formed by bending the distal ends of the first arm 211 and the second arm 212 inward.

The holding member (tubular member) 3 is a tubular member capable of storing at least a part of the clip 2. The holding member 3 has an internal space through which the clip 2 advances and retracts in the longitudinal direction A. The holding member 3 can fix the clip 2 in a closed state when it is retracted into the internal space. As shown in FIG. 5, the holding member 3 has a holding tube 3A provided on the proximal end side A2 and a holding pipe 3B provided on the distal end side A1.

The holding tube (second tubular member) 3A is formed in a cylindrical shape. The holding tube 3A is formed by injection molding a material softer than the clip 2, such as a thermoplastic resin having appropriate elasticity, such as PPA (polyphthalamide), PA (polyamide), PEEK (polyether ether ketone), or LCP (liquid crystal polymer). The holding tube 3A may be made of metal instead of thermoplastic resin.

The holding pipe (first tubular member) 3B is a cylindrical member made of metal. The holding pipe 3B is press-fitted into the distal end of the holding tube 3A. The holding tube 3A and the holding pipe 3B may be connected by heat welding, adhesive, or screw fastening.

The connecting member 4 is detachably connected to the proximal end of the clip 2. The connecting member 4 is also detachably connected to the arrowhead hook 231 that passes through the sheath 220. In other words, the connecting member 4 connects the clip 2 and the arrowhead hook 231.

### [Cartridge 5]

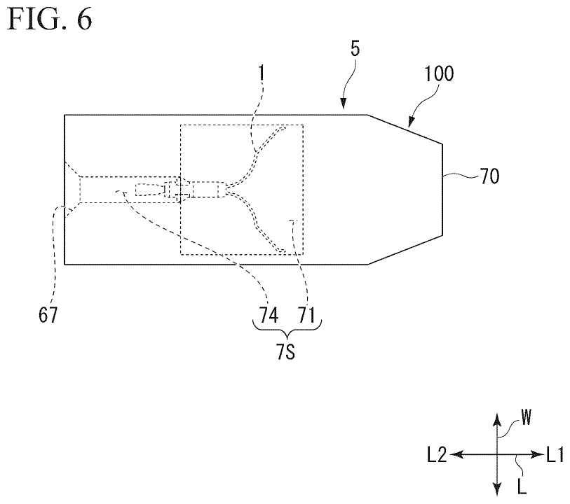

FIG. 6 is a plan view showing the cartridge 5.

A system having a clip unit 1 and a cartridge 5 capable of housing the clip unit 1 is referred to as a cartridge system 100. The cartridge system 100 is a support system for easily loading the clip unit 1 into the clip introduction device 200.

The cartridge 5 is a case for storing the clip unit 1, as shown in FIG. 6. The cartridge 5 is about 40 mm wide, 70 to 80 mm long, and 5 mm thick, and is formed to be easy to hold.

The cartridge 5 is manufactured by injection molding using a transparent resin material with appropriate hardness, such as ABS, PC, PP, PS, acrylic, or cycloolefin polymer. The cartridge 5 is formed using a transparent resin material, so that the user can easily determine whether the clip unit 1 is present inside.

As shown in FIG. 6, one of the two directions perpendicular to the longitudinal direction (insertion direction) L of the cartridge 5 and perpendicular to each other is called the “width direction W” and the other is called the “height direction H”. The plane horizontal to the longitudinal direction L and the width direction W is called the “horizontal plane HP”. The plane horizontal to the longitudinal direction L and the height direction H is defined as a “vertical plane VP.” In the cartridge 5 storing the clip unit 1, the pair of arms 21 side is defined as the distal end side L1 of the cartridge 5, and the connecting member 4 side is defined as the proximal end side L2 of the cartridge 5.

As shown in FIG. 6, the cartridge 5 has a cartridge body (case body) 70 formed in a substantially rectangular box shape. The cartridge body 70 has an insertion port (opening) 67 formed therein. A treatment tool storage area 7S is formed inside the cartridge body 70.

The cartridge body 70 has a treatment tool storage area 7S formed therein in which the clip unit 1 is stored so as to be movable in the longitudinal direction (advance and retract direction) L. The treatment tool storage area 7S includes a first area 71 and a sheath insertion area 74, as shown in FIG. 6. The first area 71 and the sheath insertion area 74 are arranged from the distal end side L1 to the proximal end side L2 in the longitudinal direction L of the cartridge 5.

The cartridge body 70 is formed with an insertion port (opening) 67 through which the sheath 220 can be inserted. The insertion port 67 has an opening surface formed substantially perpendicular to the longitudinal direction L. The insertion port 67 is formed on the side surface of the proximal end side L2 of the cartridge body 70. The insertion port 67 communicates with the sheath insertion region 74.

The first region 71 is an internal space in which the clip unit 1 is stored so as to be movable in the longitudinal direction L. The first region 71 communicates with the sheath insertion region 74.

The sheath insertion region 74 is an area into which the distal end of the sheath 220 that has passed through the insertion port 67 is inserted. The sheath insertion region 74 is located on the proximal end side L2 of the first region 71 and communicates with the first region 71.

### [Method of Loading Clip Unit 1]

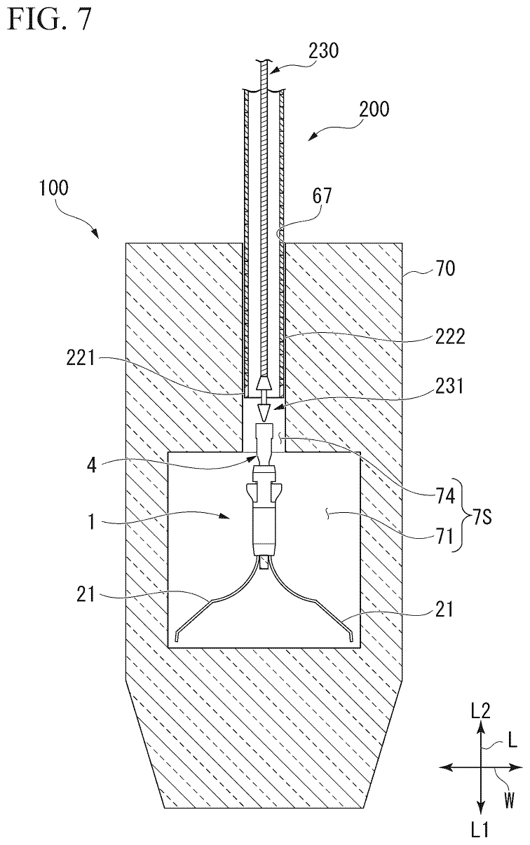

Next, a method of loading the clip unit 1 will be described with reference to FIGS. 7 to 9.

FIGS. 7 to 9 are diagrams for explaining a method of loading the clip unit 1 into the clip introduction device 200 using the cartridge 5.

FIG. 7 is a diagram showing the clip unit 1 connected to the clip introduction device 200.

The user advances the sheath 220 toward the distal end side of the cartridge 5. At this time, the user, for example, holds the cartridge 5 with one hand and moves the sheath 220 with the other hand. The user brings the arrowhead hook 231 into contact with the connecting member 4 by further advancing the sheath 220.

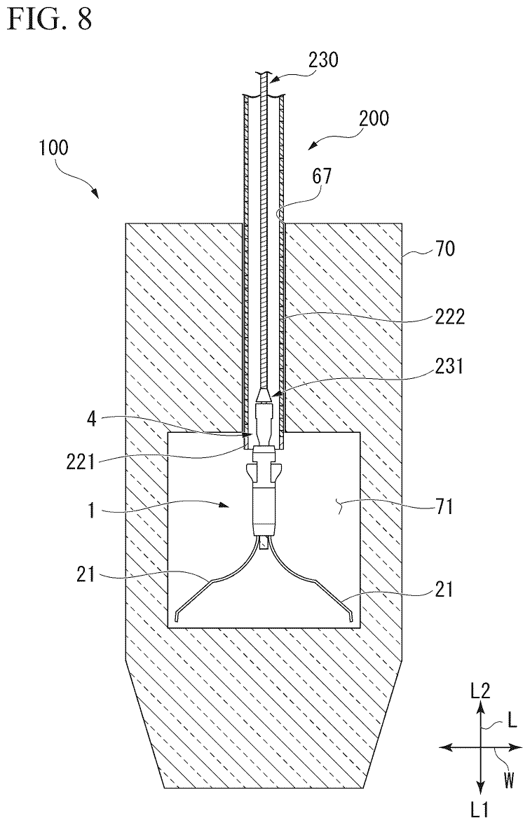

FIG. 8 is a diagram showing the clip unit 1 connected to the clip introduction device 200.

The user advances the sheath 220 further to connect the engagement portion 231a of the arrowhead hook 231 to the connecting member 4.

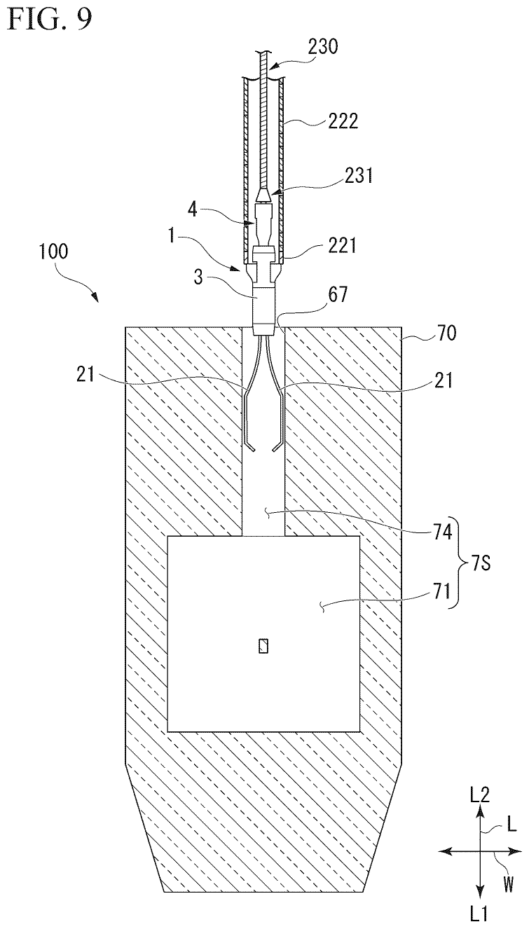

FIG. 9 shows the clip unit 1 being pulled out of the cartridge 5.

The user retracts the sheath 220 to the proximal end side L2 of the cartridge 5. At this time, the user, for example, holds the cartridge 5 with one hand while moving the sheath 220 with the other hand. The sheath 220 pulls the clip unit 1 to the proximal end side L2. By retracting the sheath 220 to the proximal end side L2 of the cartridge 5, the user can pull out the clip unit 1 loaded in the clip introduction device 200 from the cartridge 5.

The clip unit 1 pulled out of the cartridge 5 can be inserted into the treatment tool channel 430 of the endoscope 400, which will be described later, for use.

### [Endoscope System 500]

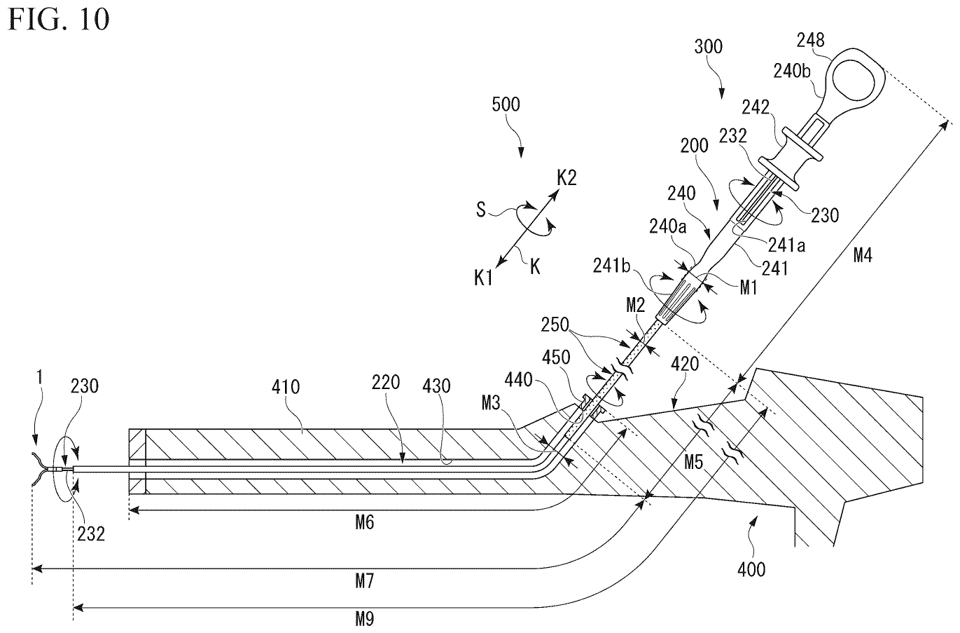

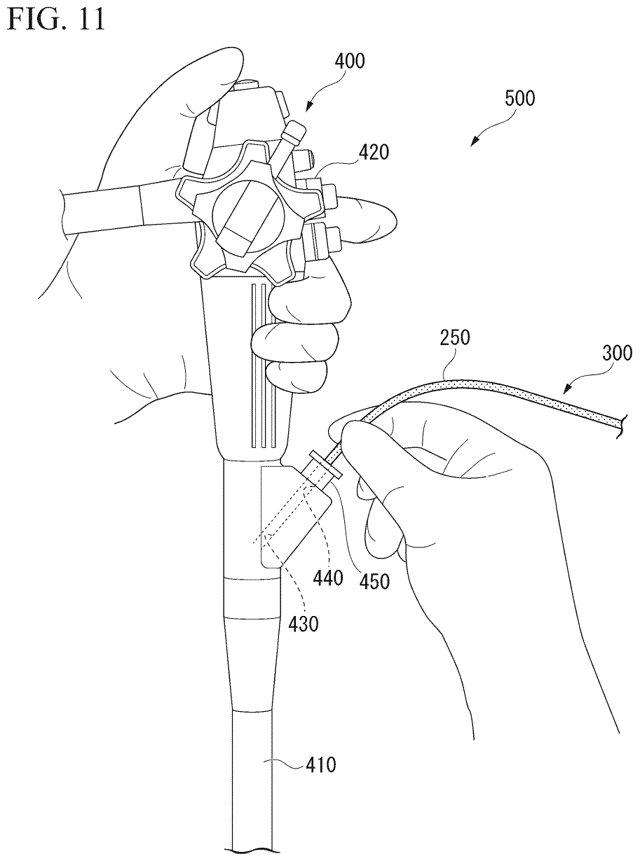

Here, the endoscope system 500 will be described with reference to FIGS. 10 and 11.

FIG. 10 shows the state in which the sheath 220 of the clip device 300 is inserted into the treatment tool channel 430 of the endoscope 400. FIG. 11 is a perspective view showing the endoscope system 500 in use.

A system having the clip device 300 and the endoscope 400 is called the endoscope system 500. The endoscope system 500 is a system used during endoscopic treatment.

The endoscope 400 is a known direct-view type flexible endoscope, and includes a long insertion portion 410, an endoscope operation portion 420, and a treatment tool channel 430. The endoscope operation portion 420 is provided at the proximal end of the insertion portion 410. In the following description, the endoscope operation portion 420 side of the endoscope 400 is referred to as the proximal side. The opposite side of the endoscope operation portion 420 in the longitudinal axis direction of the insertion portion 410 is referred to as the distal side of the endoscope 400. The treatment tool channel 430 is a member through which a treatment part such as the clip device 300 can be inserted. The treatment tool channel 430 is a member through which a treatment part such as the clip unit 1 can be inserted. The endoscope 400 may be a side-viewing type flexible endoscope.

A forceps port 440 communicating with the treatment tool channel 430 is provided distal to the endoscope operation portion 420. A user can insert an endoscopic treatment tool such as the clip device 300 from the forceps port 440. A forceps plug 450 is attached to the forceps port 440 to prevent leakage of bodily fluids.

Here, the configuration of the clip device 300 compared to the endoscope 400 will be described.

The operation portion 240 is formed so as not to be able to be inserted into the treatment tool channel 430. Specifically, the outer diameter M1 of the operation portion 240 is larger than the inner diameter M3 of the treatment tool channel 430. The outer sheath 250 is formed so as to be able to be inserted into the treatment tool channel 430. Specifically, the outer diameter M2 of the outer sheath 250 is smaller than the inner diameter M3 of the treatment instrument channel 430.

The length M7 from the distal end of the clip unit 1 to the distal end of the outer sheath 250 in the longitudinal direction K of the clip device 300 is shorter than the length M6 of the treatment instrument channel 430. Specifically, when the sheath 220 is inserted into the treatment instrument channel 430 and the clip unit 1 protrudes from the distal end of the treatment instrument channel 430, the distal end of the outer sheath 250 is contained in the treatment instrument channel 430.

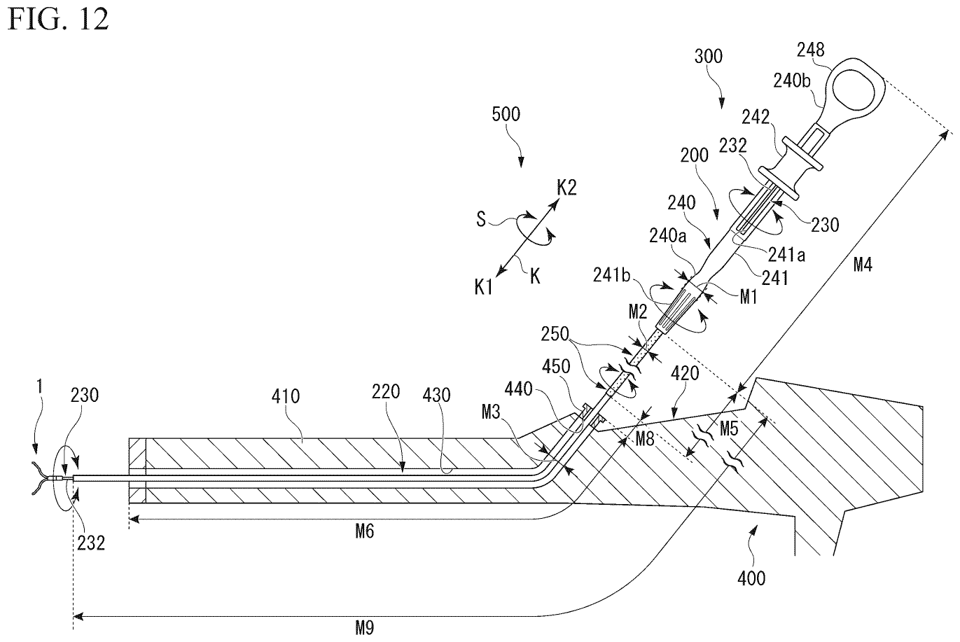

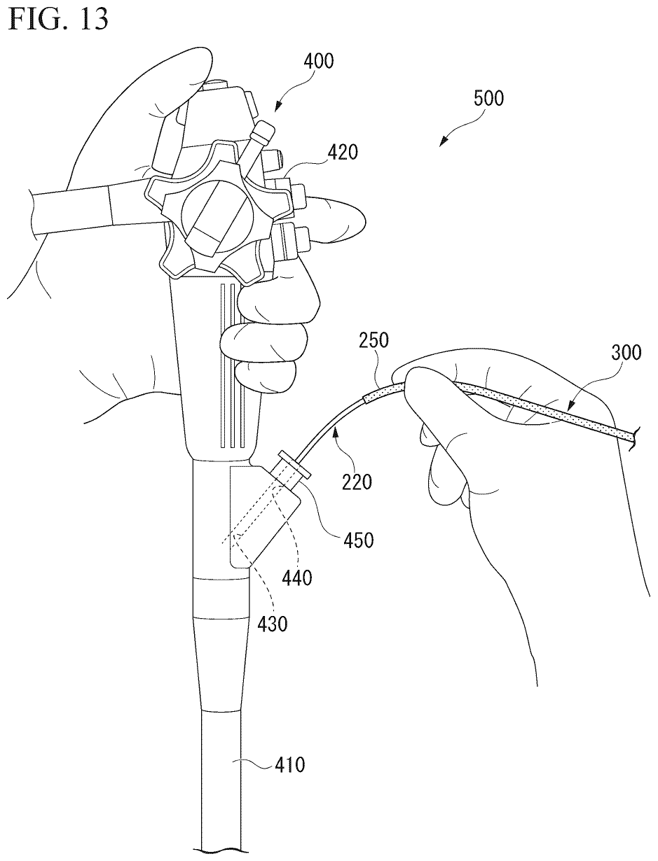

As shown in FIGS. 12 and 13, when the clip unit 1 protrudes from the distal end of the treatment instrument channel 430, the distal end of the outer sheath 250 does not have to be contained in the treatment instrument channel 430. Specifically, when the clip unit 1 protrudes from the distal end of the treatment tool channel 430, the length M8 between the distal end of the outer sheath 250 and the forceps port 440 may be 30 cm or less. In that case, the user can rotate and move forward and backward at the vicinity of the forceps port 440 while grasping one point of the outer sheath 250, and the user can operate it intuitively.

### [Function of the Clip Device 300]

Next, the function of the clip device 300 will be explained with reference to FIGS. 10 and 11.

As shown in FIG. 10, the clip device 300 is used with the sheath 220 inserted into the treatment tool channel 430. The user inserts the sheath 220 until the clip unit 1 protrudes from the treatment tool channel 430. At this time, the distal end of the outer sheath 250 is accommodated in the treatment tool channel 430.

As shown in FIG. 11, the user operates the endoscope operation portion 420 with one hand and operates the outer sheath 250 with the other hand. When using the endoscope system 500, a caregiver who operates the operation portion 240 of the clip device 300 is required in addition to the user who operates the endoscope 400.

The user needs to rotate the clip unit 1 appropriately. The user can rotate the clip unit 1 in the circumferential direction S by rotating the outer sheath 250 in the circumferential direction S. When the outer sheath 250 rotates in the circumferential direction S, the rotation is transmitted in the order of the operation portion main body 241 and the operation wire 230, and the clip unit 1 rotates in the circumferential direction S. In other words, a force to rotate the outer sheath 250 can be transmitted to the treatment portion via the operation portion 240 and the operation wire 230, and the outer sheath 250 itself has the rigidity to transmit a rotational force to the operation portion 240. By rotating the clip unit 1 in the circumferential direction S, the user can operate the clip unit 1 in an appropriate orientation.

According to the clip device 300 of this embodiment, the clip unit 1 can be rotated by rotating the outer sheath 250, so that the user can rotate the clip unit 1 in the circumferential direction S while operating the endoscope 400. Specifically, the user can rotate the clip unit 1 in the circumferential direction S by operating the outer sheath 250 with the other hand while operating the endoscope operation portion 420 with one hand. Accordingly, the clip device 300 is easy to use.

According to the clip device 300 of this embodiment, the operation wire 230, which is a member that transmits the rotation of the outer sheath 250 to the clip unit 1, is not subjected to friction from the treatment instrument channel 430, so that the rotation is easily transmitted. Specifically, since the operating wire 230 is inserted through the sheath 220, it can rotate without being subjected to friction from the treatment tool channel 430. Therefore, when the sheath 220 is inserted through the treatment tool channel 430, the clip unit 1 can be rotated easily, and treatment using the clip unit 1 can proceed smoothly.

According to the clip device 300 according to this embodiment, since the outer sheath 250 can rotate relative to the sheath 220, when the outer sheath 250 is rotated in the circumferential direction S, the sheath 220 does not rotate in the circumferential direction S, and is not affected by friction between the sheath 220 and the treatment tool channel 430. Therefore, when the sheath 220 is inserted through the treatment tool channel 430, the clip unit 1 can be rotated easily, and treatment using the clip unit 1 can proceed smoothly.

In the clip device 300 according to this embodiment, the outer sheath 250 is formed in a cylindrical shape through which the sheath 220 is inserted, so that the user can easily rotate the outer sheath 250 in the circumferential direction S. Therefore, when the sheath 220 is inserted into the treatment tool channel 430, the clip unit 1 can be easily rotated, and treatment using the clip unit 1 can be smoothly performed.

In the clip device 300 according to this embodiment, the outer sheath 250 has higher flexibility than the operation portion 240 and can be bent along the sheath 220, so that the shape of the sheath 220 can be deformed while the clip unit 1 can be rotated at a position away from the operation portion 240. Therefore, the clip device 300 is easy to use.

According to the clip device 300 of this embodiment, the outer diameter M2 of the outer sheath 250 is smaller than the inner diameter M3 of the treatment tool channel 430, so the outer sheath 250 can be used while inserted into the treatment tool channel 430. This prevents the distal end of the outer sheath 250 from coming into contact with the forceps port 440 while operating the clip device 300, which can hinder operation, and the clip device 300 is easy to use.

According to the clip device 300 of this embodiment, the length M5 of the outer sheath 250 in the longitudinal direction K is longer than the length M4 of the operation portion 240, so the user can operate the clip unit 1 at a position away from the operation portion 240. This makes the clip device 300 easy to use.

According to the clip device 300 of this embodiment, the length M5 of the outer sheath 250 in the longitudinal direction K is shorter than the length M9 of the sheath 220, so when the sheath 220 is inserted into the treatment instrument channel 430, the length by which the outer sheath 250 is inserted into the treatment instrument channel 430 is short. Therefore, when the sheath 220 is inserted into the treatment instrument channel 430, the clip unit 1 can be easily rotated, and treatment using the clip unit 1 can be smoothly performed.

According to the clip device 300 of this embodiment, the length M7 from the distal end of the clip unit 1 to the distal end of the outer sheath 250 in the longitudinal direction K is shorter than the length M6 of the treatment instrument channel 430, so the outer sheath 250 can be used in a state where it is inserted into the treatment instrument channel 430. Even when the distal end of the treatment instrument channel 430 and the distal end of the clip unit 1 are aligned, the distal end of the outer sheath 250 is inserted into the treatment instrument channel 430. Therefore, causes that hinder operation, such as the distal end of the outer sheath 250 coming into contact with the forceps port 440 when operating the clip device 300, can be suppressed, and the clip device 300 is easy to use.

According to the clip device 300 of this embodiment, when the sheath 220 is inserted into the treatment tool channel 430 and the clip unit 1 protrudes from the distal end of the treatment tool channel 430, the distal end of the outer sheath 250 is accommodated in the treatment tool channel 430, so the outer sheath 250 can be used while inserted into the treatment tool channel 430. Therefore, causes that hinder operation, such as the distal end of the outer sheath 250 coming into contact with the forceps port 440 when operating the clip device 300, can be suppressed, and the clip device 300 is easy to use.

In the clip device 300 according to this embodiment, the operation portion 240 has a first part 240a that can rotate in conjunction with the outer sheath 250, and a second part 240b that is connected to the first part 240a so as to be rotatable relative to the first part 240a. Therefore, when the user rotates the outer sheath 250 in the circumferential direction S and the first part 240a rotates in the circumferential direction S, the caregiver operating the operation portion 240 supports the second part 240b and does not impede the rotation of the first part 240a. Therefore, when the sheath 220 is inserted into the treatment tool channel 430, the clip unit 1 can be easily rotated, and treatment using the clip unit 1 can be smoothly performed.

In the clip device 300 according to this embodiment, the slider 242 is disposed in the first part 240a. Therefore, the caregiver operating the operation portion 240 can easily move the slider 242 forward and backward in the longitudinal direction K, and the clip device 300 is easy to use.

According to the clip device 300 of this embodiment, the outer sheath 250 has an inner layer 251, an outer layer 252, and a middle layer 253 that is disposed radially between the inner layer 251 and the outer layer 252 and has a higher hardness than the inner layer 251 and the outer layer 252. Therefore, the outer sheath 250 can transmit rotations applied to the distal end to the operation portion 240. Therefore, when the sheath 220 is inserted into the treatment tool channel 430, the clip unit 1 can be easily rotated, and treatment using the clip unit 1 can be smoothly performed.

According to the clip device 300 of this embodiment, the inner layer 251 is formed containing resin, the outer layer 252 is formed containing resin, and the middle layer 253 is formed containing metal wire. Therefore, the outer sheath 250 has a suitable hardness and can transmit rotations applied to the distal end to the operation portion 240. Therefore, when the sheath 220 is inserted into the treatment tool channel 430, the clip unit 1 can be easily rotated, and treatment using the clip unit 1 can be smoothly performed.

### First Modification

### [Operation Portion 240B]

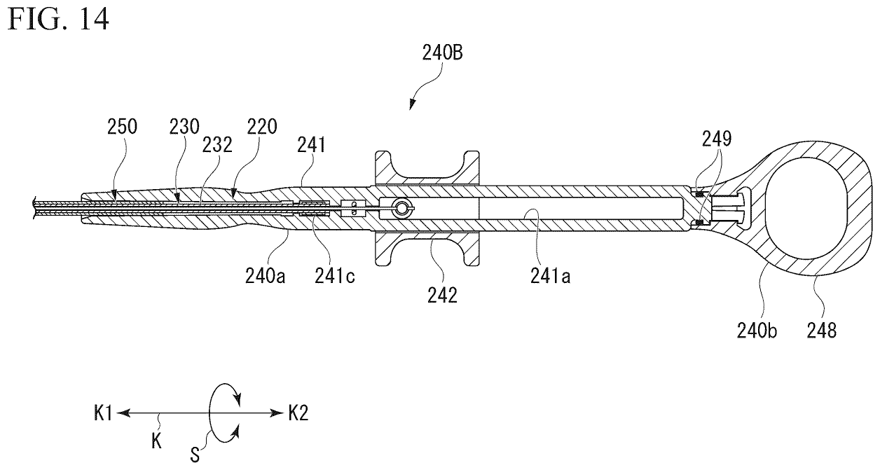

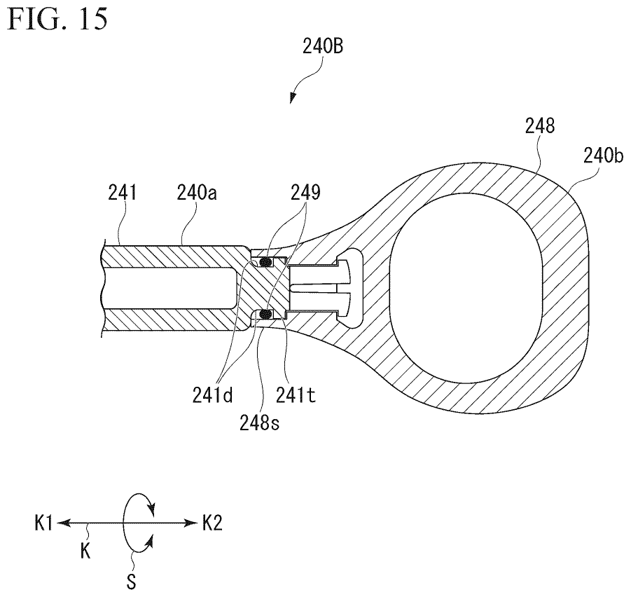

Next, the operation portion 240B according to a first modification will be described with reference to FIGS. 14 and 15.

FIG. 14 is a cross-sectional view of the operation portion 240B according to the first modification, showing a cross-section along the longitudinal direction K.

The operation portion 240B is a modification of the operation portion 240. The operation portion 240B differs from the operation portion 240 in that it has a stopper 249.

The stopper 249 is a member that restricts the relative rotation between the first part 240a and the second part 240b. In the operation portion 240B, the stopper 249 is a member that restricts the relative rotation between the operation portion main body 241 and the thumb ring 248. The stopper 249 is, for example, an O-ring. As shown in FIG. 14, the stopper 249 is provided at the connection between the operation portion main body 241 and the thumb ring 248.

FIG. 15 is an enlarged view of the area indicated by dashed line D13 of the operation portion 240B shown in FIG. 14.

As shown in FIG. 15, a radially recessed groove 241d is formed in the proximal end 241t of the operation portion main body 241. The groove 241d is formed on the outer periphery of the proximal end 241t of the operation portion main body 241. The stopper 249 is formed in a ring shape and is fitted into the groove 241d. The distal end end 248s of the thumb ring 248 is formed in a cylindrical shape and is attached so as to cover the outer periphery of the stopper 249. The stopper 249 is pressed radially by the outer circumferential surface of the proximal end 241t of the operation portion main body 241 and the inner circumferential surface of the distal end end 248s of the thumb ring 248.

The stopper 249 is a friction member that generates friction between itself and the first part 240a. In the operation portion 240B, the stopper 249 is a friction member that generates friction between itself and the operation portion main body 241. The stopper 249 is a friction member that generates friction between itself and the second part 240b. In the operation portion 240B, the stopper 249 is a friction member that generates friction between itself and the thumb ring 248. The stopper 249 is formed to contain rubber.

The friction between the stopper 249 and the operation portion main body 241 is determined according to the rotation transmission performance of the outer sheath 250. Specifically, “the friction force between the stopper 249 and the operation portion main body 241”>{rotation angle of the outer sheath 250×(1−rotation transmission rate of the outer sheath 250)}×“torque required to rotate the outer sheath 250 by 1 degree (N·mm/deg)”. For example, when the rotation operation angle of the outer sheath 250 is 90°, the rotation transmission rate of the outer sheath 250 is 33%, and the torque required to rotate the outer sheath 250 is 10 N·mm/deg, a twist of 60° accumulates in the outer sheath 250, and therefore the friction force of the stopper 249 is greater than, for example, 600 N·mm. The friction between the stopper 249 and the operation portion main body 241 and between the stopper 249 and the thumb ring 248 is preferably greater than 10 mN·m. The friction between the stopper 249 and the operation portion main body 241, and between the stopper 249 and the thumb ring 248 may be 5 mN·m or more, or 10 mN·m or less.

When the outer sheath 250 rotates in the circumferential direction S while inserted into the treatment instrument channel 430, friction occurs between the outer sheath 250 and the forceps plug 450. Therefore, if the user rotates the outer sheath 250 in the circumferential direction S with a force smaller than the frictional force between the outer sheath 250 and the forceps plug 450, the outer sheath 250 and the forceps plug 450 do not rotate relative to each other in the circumferential direction S. Even if the outer sheath 250 and the forceps plug 450 do not rotate relative to each other in the circumferential direction S, the clip unit 1 rotates via the operation portion 240 and the operating wire 230 due to the outer sheath 250 twisting. However, when the outer sheath 250 is untwisted, the clip unit 1 returns to its original state. Therefore, the user needs to rotate the outer sheath 250 in the circumferential direction S with a force larger than the frictional force between the outer sheath 250 and the forceps plug 450.

Since the operation portion 240B has the stopper 249, the user can rotate the outer sheath 250 in the circumferential direction S with a force equal to or greater than that restricted by the stopper 249, thereby preventing the clip unit 1 from rotating due to twisting of the outer sheath 250. Therefore, when the sheath 220 is inserted into the treatment tool channel 430, the clip unit 1 can be easily rotated, and treatment using the clip unit 1 can be performed smoothly.

Since friction is generated between the operation portion main body 241 and the stopper 249, and friction is generated between the thumb ring 248 and the stopper 249, the user can rotate the outer sheath 250 in the circumferential direction S with a force equal to or greater than the frictional force between the operation portion main body 241 and the stopper 249 and the frictional force between the thumb ring 248 and the stopper 249, thereby preventing the clip unit 1 from rotating due to twisting of the outer sheath 250. Therefore, when the sheath 220 is inserted into the treatment tool channel 430, the clip unit 1 can be easily rotated, and treatment using the clip unit 1 can be smoothly performed.

When a force greater than a predetermined amount is applied to the operation portion main body 241 and the treatment tool channel 430 in the circumferential direction S, the operation portion main body 241 and the treatment tool channel 430 rotate relative to each other in the circumferential direction S, so the user rotates the outer sheath 250 in the circumferential direction S with a force greater than or equal to the frictional force between the operation portion main body 241 and the stopper 249 and the frictional force between the thumb ring 248 and the stopper 249. Therefore, it is possible to prevent the clip unit 1 from rotating due to twisting of the outer sheath 250, and when the sheath 220 is inserted into the treatment tool channel 430, the clip unit 1 can be easily rotated, and treatment using the clip unit 1 can be smoothly performed.

The stopper 249 shown in the first modified example is formed in a ring shape, but the shape of the stopper 249 is not limited. The stopper 249 may be configured to restrict the relative rotation between the operation portion main body 241 and the thumb ring 248.

### Second Modification

### [Operation Portion 240C]

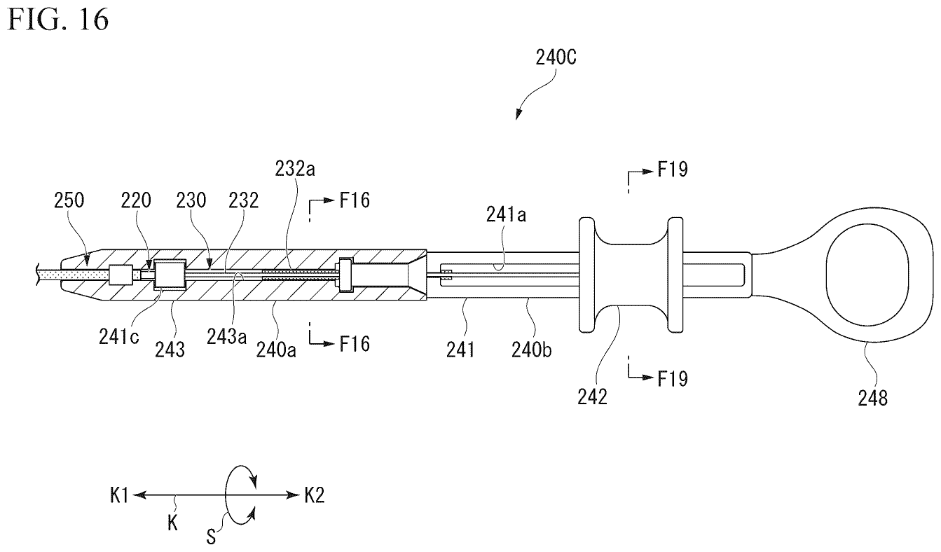

Next, the operation portion 240C according to a second modification will be described with reference to FIGS. 16 to 22.

FIG. 16 is a side view of the operation portion 240C according to the second modification, with a partial cross section.

The operation portion 240C is a modification of the operation portion 240. The operation portion 240C differs from the operation portion 240 in that it has a rotation operation portion 243.

The rotation operation portion 243 is a member that is provided so as to be rotatable relative to the operation portion main body 241 in the circumferential direction S. The rotation operation portion 243 is provided on the distal end side of the operation portion main body 241 as shown in FIG. 16. The rotation operation portion 243 rotates in the circumferential direction S relative to the operation portion main body 241.

As shown in FIG. 16, the sheath 220 is connected to the rotating operation portion 243 via a retainer 241c. The sheath 220 is connected to the rotating operation portion 243 so as not to be able to advance or retract in the longitudinal direction K. The sheath 220 is connected to the rotating operation portion 243 so as to be able to rotate in the circumferential direction S.

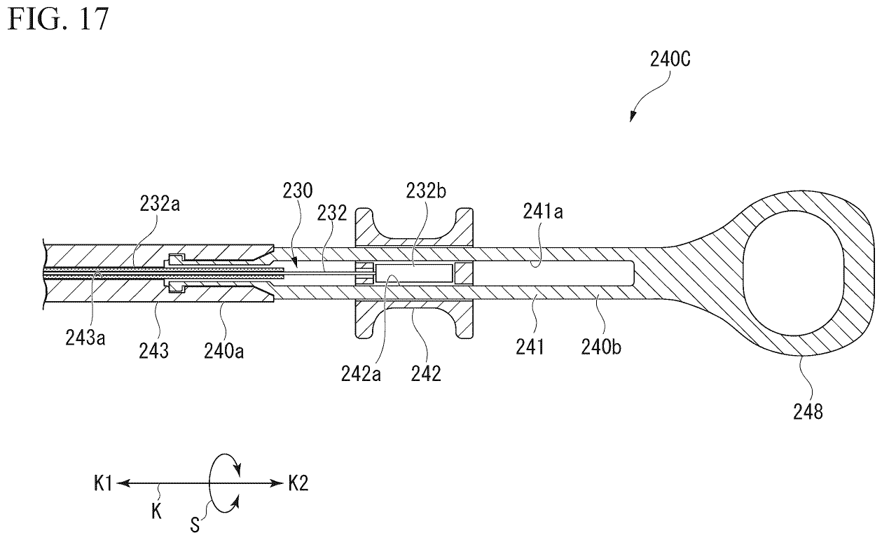

FIG. 17 is a cross-sectional view of the operation portion 240C, showing a cross-section along the longitudinal direction K. As shown in FIG. 16 and FIG. 17, the wire 232 passes through the insertion passage 243a formed inside the rotating operation portion 243 toward the proximal end side, and is then connected to the slider 242.

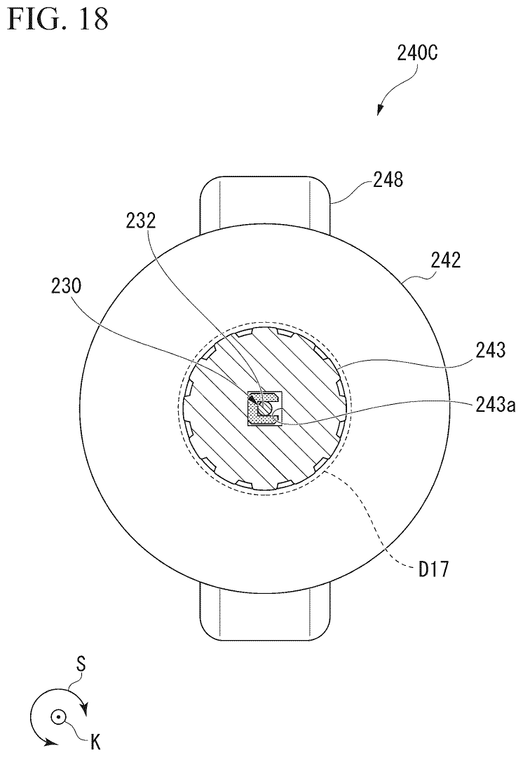

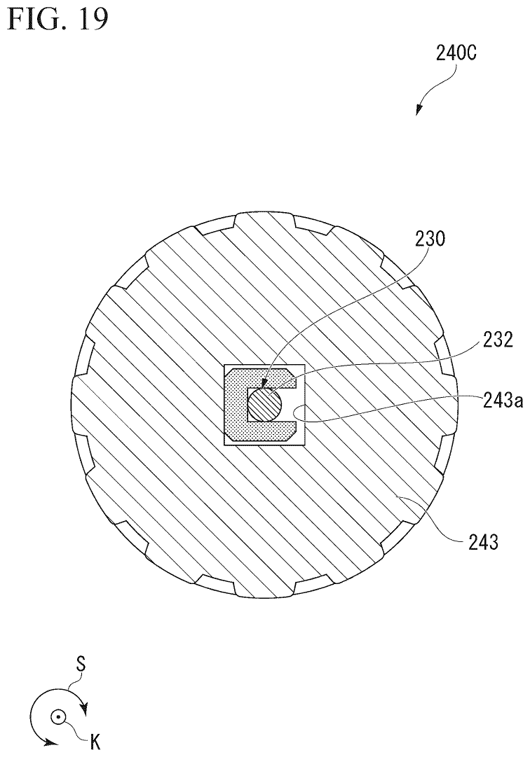

FIG. 18 is a cross-sectional view along the line F14-F14 of the operation portion 240C shown in FIG. 16. FIG. 19 is an enlarged view of the range indicated by the dashed line D17 of the operation portion 240C shown in FIG. 18. The wire 232 is provided so as to be movable forward and backward in the longitudinal direction K relative to the rotation operation portion 243. The wire 232 is provided so as to be rotatable in the circumferential direction S relative to the operation portion main body 241 in conjunction with the rotation operation portion 243.

The wire 232 is provided with a rotation transmission member 232a that transmits the rotation of the rotation operation portion 243 in the circumferential direction S to the wire. As shown in FIGS. 17 to 19, the rotation transmission member 232a is provided on the outer periphery of the wire 232. The rotation transmission member 232a is provided in a region of the outer periphery of the wire 232 that passes through the insertion passage 243a in the longitudinal direction K. The rotation transmission member 232a is fixed to the wire 232.

The rotation transmission member 232a is a member that extends in the longitudinal direction K. The rotation transmission member 232a is formed in a rotationally asymmetric shape when viewed from the longitudinal direction K. The rotation transmission member 232a is formed, for example, in a U-shape when viewed from the longitudinal direction K.

The insertion passage 243a through which the rotation transmission member 232a is inserted is formed in a shape that restricts the rotation of the rotation transmission member 232a in the circumferential direction S when viewed from the longitudinal direction K. The insertion passage 243a is formed in a rotationally asymmetric shape when viewed from the longitudinal direction K. The insertion passage 243a is formed, for example, in a substantially rectangular shape. Therefore, the rotation transmission member 232a can transmit the rotation of the rotation operation portion 243 in the circumferential direction S to the wire while being able to advance and retract through the insertion passage 243a in the longitudinal direction K.

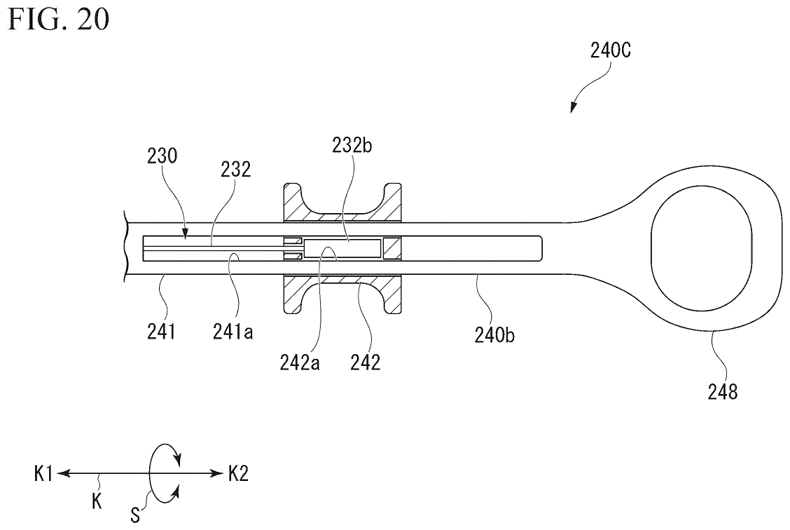

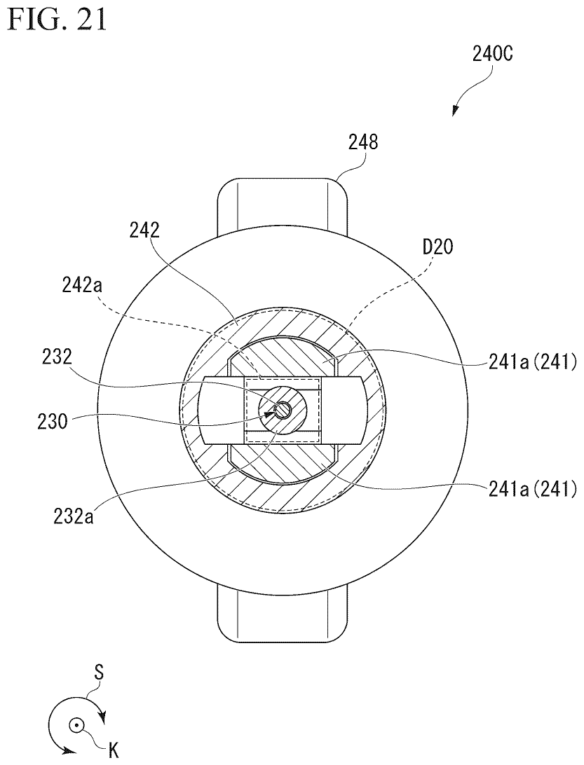

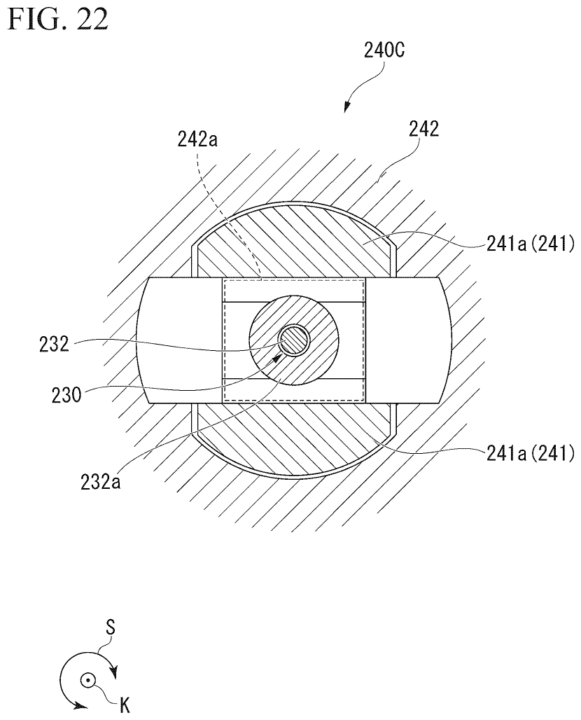

FIG. 20 is a side view showing the operation portion main body 241, showing a cross section of the slider 242. FIG. 21 is a cross section along the F19-F19 line of the operation portion 240C shown in FIG. 16. FIG. 22 is an enlarged view of the area indicated by the dashed line D20 of the operation portion 240C shown in FIG. 21.

The wire 232 is provided so as to be movable back and forth in the longitudinal direction K in conjunction with the slider 242. The wire 232 is provided so as to be rotatable in the circumferential direction S relative to the slider 242.

The wire 232 is provided with a stopper 232b that transmits the movement of the slider 242 back and forth in the longitudinal direction K to the wire 232. The stopper 232b is provided on the outer periphery of the wire 232 as shown in FIG. 20 to FIG. 22. The stopper 232b is provided in an area of the outer periphery of the wire 232 that overlaps with the slider 242 in the longitudinal direction K. The stopper 232b is provided at the proximal end of the wire 232. The stopper 232b is fixed to the wire 232.

The retaining member 232b is a member extending in the longitudinal direction K. When viewed from the longitudinal direction K, the retaining member 232b is formed in a circular shape.

The retaining member 232b is accommodated in a storage space 242a formed inside the slider 242. The length of the storage space 242a in the longitudinal direction K is slightly longer than the length of the retaining member 232b in the longitudinal direction K, as shown in FIG. 20. Therefore, the movement of the retaining member 232b in the longitudinal direction K relative to the storage space 242a is restricted, and the advancement and retract of the slider 242 in the longitudinal direction K is transmitted to the wire 232. When viewed from the longitudinal direction K, the storage space 242a is formed to a size that allows the retaining member 232b to rotate in the circumferential direction S, as shown in FIG. 21 and FIG. 22. Specifically, the length of the longest part of the width of the retainer 232b as viewed from the longitudinal direction K should be shorter than the length of the shortest part of the width of the accommodation space 242a. For example, the accommodation space 242a is formed in a substantially rectangular shape formed by sides larger than the diameter of the retainer 232b as viewed from the longitudinal direction K. Therefore, the retainer 232b can transmit the advancement and retract of the slider 242 in the longitudinal direction K to the wire 232 while being rotatable in the circumferential direction S.

In the operation portion 240C, the thumb ring 248 is fixed to the operation portion main body 241.

Similar to the operation portion 240, the operation portion 240C has a first part 240a connected to the outer sheath 250 and a second part 240b connected to the first part 240a so as to be rotatable relative to the first part 240a in the circumferential direction S. In the operation portion 240C, the first part 240a has a rotation operation portion 243. In the operation portion 240C, the second part 240b has an operation portion main body 241, a slider 242, and a thumb ring 248.

With the operation portion 240C, when the outer sheath 250 rotates in the circumferential direction S, the rotation operation portion 243 rotates in the circumferential direction S in conjunction with the operation portion, but the operation portion main body 241 and the slider 242 do not rotate in conjunction with the operation portion. Therefore, it is easy for a caregiver operating the operation portion 240C to maintain the position of the slider 242. The clip device 300 equipped with the operation portion 240C is easy to use.

The operation portion 240C has a rotation transmission member 232a and a stopper 232b, but the configuration of the operation portion 240C is not limited. The operation portion 240C may have a configuration in which the first part 240a has a rotating operation portion 243, and the second part 240b has an operation portion main body 241, a slider 242, and a thumb ring 248.

### Third Modification

### [Operation Portion 240D]

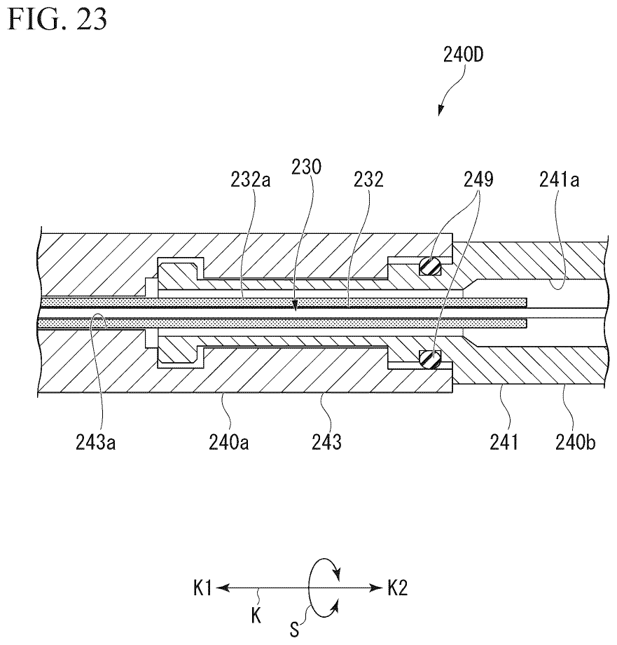

Next, the operation portion 240D according to a third modification will be described with reference to FIG. 23.

FIG. 23 is a cross-sectional view of the operation portion 240D according to the third modification, showing a cross-section along the longitudinal direction K.

The operation portion 240D is a modification of the operation portion 240C. The operation portion 240D differs from the operation portion 240 in that it has a stopper 249.

In the operation portion 240D, the stopper 249 is a member that restricts the relative rotation in the circumferential direction S between the rotation operation portion 243 and the operation portion main body 241. The stopper 249 is provided at the connection portion between the rotation operation portion 243 and the operation portion main body 241, as shown in FIG. 23.

With the operation portion 240D, the user rotates the outer sheath 250 in the circumferential direction S with a force equal to or greater than that restricted by the stopper 249, and therefore it is possible to prevent the clip unit 1 from rotating due to twisting of the outer sheath 250. Therefore, when the sheath 220 is inserted into the treatment instrument channel 430, it is easy to rotate the clip unit 1, and treatment using the clip unit 1 can be smoothly performed.

### Fourth Modification

### [Outer Sheath 250E]

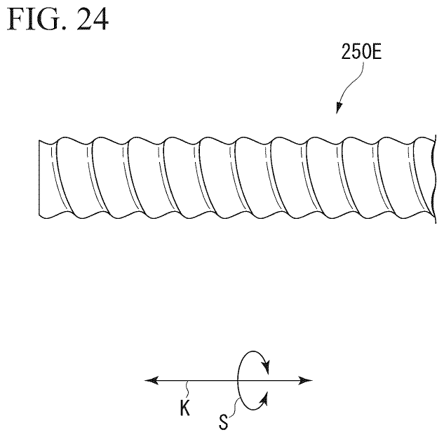

Next, the outer sheath 250E according to a fourth modification will be described with reference to FIGS. 24 to 26.

FIG. 24 is a side view showing the outer sheath 250E according to the fourth modification.

The outer sheath 250E is a modification of the outer sheath 250. The outer sheath 250E is configured to be extendable and contractible in the longitudinal direction K.

The outer sheath 250E is a member that can transition between a contracted state and an extended state. The contracted state is a state in which the outer sheath 250E has a first length in the longitudinal direction K. The extended state is a state in which the outer sheath 250E has a second length in the longitudinal direction K that is longer than the first length. The outer sheath 250E is configured in a bellows shape, for example, as shown in FIG. 24. The outer sheath 250E can rotate in the circumferential direction S relative to the sheath 220 regardless of whether the outer sheath 250E is in the contracted state or the extended state.

The outer sheath 250E is configured to be expandable and contractible, so that the length of the outer sheath 250E in the longitudinal direction K can be adjusted. Therefore, the clip device 300 including the outer sheath 250E is easy to use.

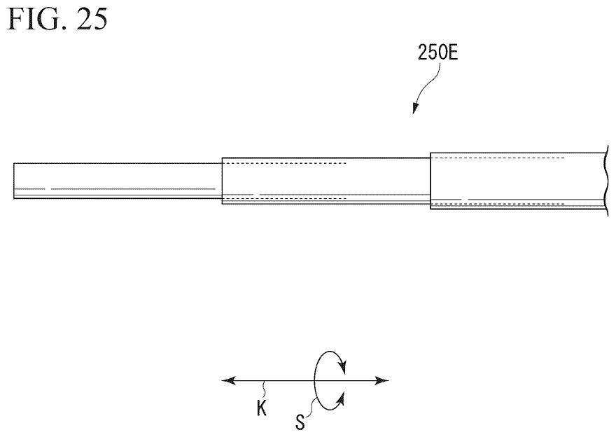

FIG. 25 is a side view showing the outer sheath 250E configured to be expandable and contractible. The configuration of the outer sheath 250E is not limited. As shown in FIG. 25, the outer sheath 250E may be configured with a plurality of cylindrical members having different radial sizes connected in the longitudinal direction K.

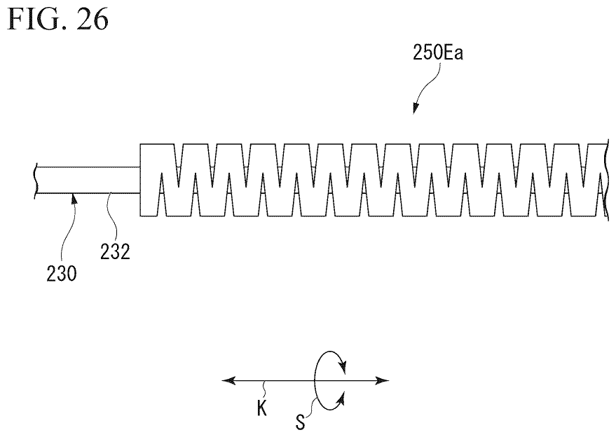

FIG. 26 is a side view showing a transmission member 250Ea configured to be expandable and contractable in the longitudinal direction K.

Instead of the outer sheath 250E, the clip device 300 may be provided with a transmission member 250Ea configured to be expandable and contractable in the longitudinal direction K as shown in FIG. 26. The transmission member 250Ea shown in FIG. 26 is a member having a slit formed in a plate-like member that is elastically deformable.

In the clip device 300 according to this embodiment, the outer sheath 250 is provided, but it is sufficient that a transmission member that is fixed to the operation portion 240 and can transmit rotation in the circumferential direction S to the operation portion 240 is provided.

In the clip device 300 according to this embodiment, the outer sheath 250 has an inner layer 251, an outer layer 252, and a middle layer 253, but the configuration of the outer sheath 250 is not limited. The outer sheath 250 may be, for example, a single-layer tube. The outer sheath 250 may be, for example, a tubing coil. The outer sheath 250 may have any configuration as long as it is capable of transmitting rotation in the circumferential direction S to the operation portion 240.

In the clip device 300 according to this embodiment, the clip unit 1 is loaded into the clip unit 1 using the cartridge 5, but the configuration of the clip unit 1 is not limited. The clip unit 1 may be attached to the clip introduction device 200 in advance. Also, a new clip unit 1 may be reloaded into the clip introduction device 200 using the cartridge 5.

In this embodiment, the treatment portion is the clip unit 1, but the treatment portion is not limited to this. The treatment portion may be any member that is attached to the clip introduction device 200 and used in endoscopic treatment, such as forceps or a knife.

In this embodiment, the clip device 300 is used as an example of a medical device, but the clip introduction device 200 can be used as an applicator in a medical device in which a treatment portion such as forceps or a knife is connected to an applicator.

The first embodiment of the present disclosure has been described above in detail with reference to the drawings, but the specific configuration is not limited to this embodiment, and design changes that do not deviate from the gist of the present disclosure are also included. In addition, the components shown in the above-mentioned embodiments and modified examples can be appropriately combined to form a configuration.

### Second Embodiment

### [Clip Device 300F]

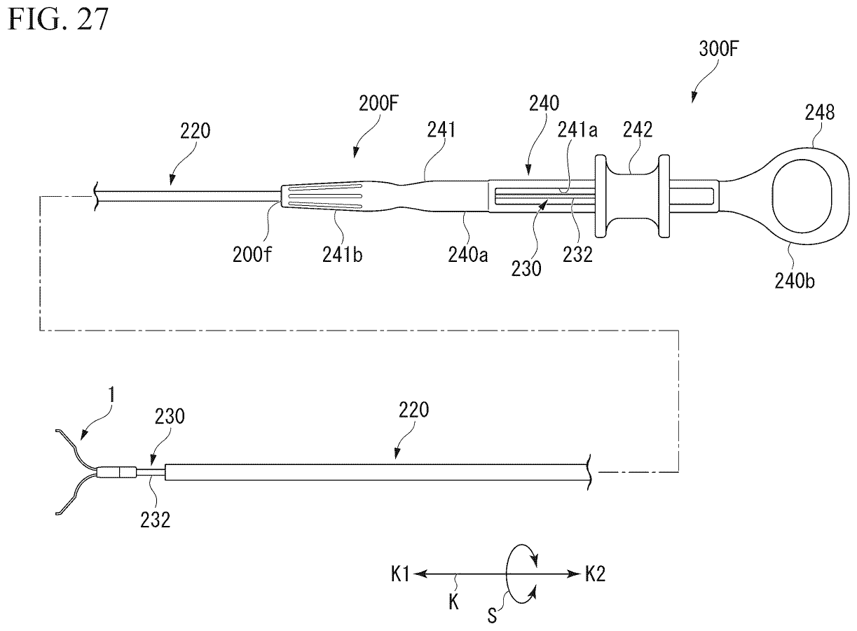

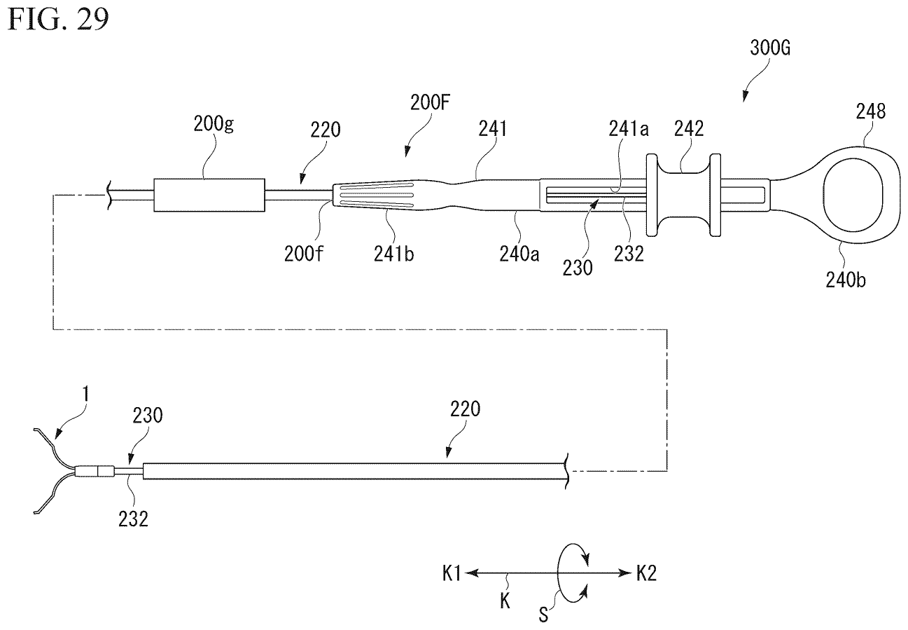

A clip device 300F according to a second embodiment of the present disclosure will be described with reference to FIGS. 27 and 29. The clip device 300F according to the second embodiment has a different configuration of the clip introduction device compared to the clip device 300 according to the first embodiment. In the following description, components common to those already described will be given the same reference numerals and redundant description will be omitted.

FIG. 27 is a side view showing a clip device 300F according to the second embodiment.

The clip device 300F includes a clip introducing device 200F and a clip unit 1. The clip unit 1 is loaded into the clip introducing device 200F.

The clip unit 1 and the sheath 220 are not fixed. Furthermore, the clip unit 1 can rotate relative to the sheath 220 in the circumferential direction S.

### [Clip Introducing Device 200F]

The clip introducing device 200F includes a sheath 220, an operating wire 230, and an operation portion 240. Unlike the clip introducing device 200, the clip introducing device 200F does not need to include an outer sheath 250.

In the clip introduction device 200F, the sheath 220 and the operation portion 240 are fixed. The connection section 200f between the sheath 220 and the operation portion 240 is fixed, for example, by adhesive.

When the sheath 220 rotates in the circumferential direction S, the operation portion 240 also rotates in the circumferential direction S in conjunction with the sheath 220. The clip unit 1, the operating wire 230, the operation portion 240, and the sheath 220 can rotate in the circumferential direction S in conjunction with each other. Therefore, the user can rotate the clip unit 1 via the operation portion 240 and the operating wire 230 by rotating the sheath 220 in the circumferential direction S.

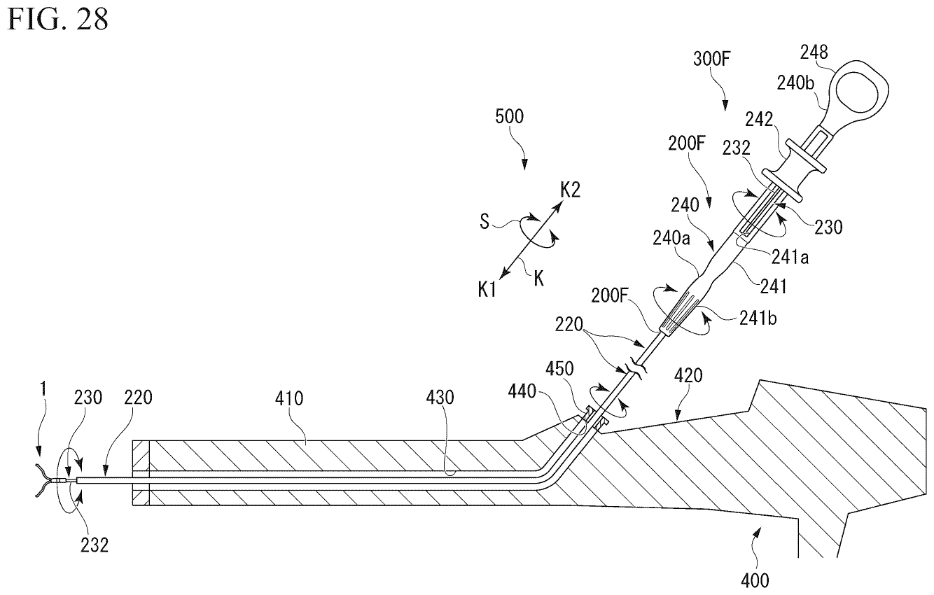

FIG. 28 shows the state in which the sheath 220 of the clip device 300F is inserted into the treatment tool channel 430 of the endoscope 400.

As shown in FIG. 28, the clip device 300F is used with the sheath 220 inserted into the treatment tool channel 430. The user inserts the sheath 220 until the clip unit 1 protrudes from the treatment tool channel 430.

The user operates the endoscope operation portion 420 with one hand and operates the sheath 220 with the other hand. In addition to the user who operates the endoscope 400, a caregiver is required to operate the operation portion 240 of the clip device 300F.

The user needs to rotate the clip unit 1 appropriately. The user can rotate the clip unit 1 by rotating the sheath 220 in the circumferential direction S. When the sheath 220 rotates in the circumferential direction S, the rotation is transmitted in the order of the operation portion main body 241 and the operation wire 230, and the clip unit 1 rotates. By rotating the clip unit 1, the user can operate the clip unit 1 in an appropriate orientation.

According to the clip device 300F of this embodiment, the clip unit 1 can be rotated by rotating the sheath 220 in the circumferential direction S, so that the user can rotate the clip unit 1 while operating the endoscope 400. Specifically, the user can rotate the clip unit 1 by operating the outer sheath 250 with one hand while operating the endoscope operation portion 420 with the other hand. Therefore, the clip device 300F is easy to use.

According to the clip device 300F of this embodiment, the operation wire 230, which is a member that transmits the rotation of the sheath 220 in the circumferential direction S to the clip unit 1, is not subjected to friction from the treatment instrument channel 430, so that the rotation is easily transmitted. Specifically, since the operation wire 230 is inserted through the sheath 220, it can rotate in the circumferential direction S without friction from the treatment tool channel 430. Although the sheath 220 is inserted through the treatment tool channel 430, as shown in FIG. 28, the portion that transmits the rotation in the circumferential direction S to the operation portion 240 is exposed from the treatment tool channel 430, so that when the rotation in the circumferential direction S of the sheath 220 is transmitted to the operation portion 240, it is not easily affected by friction between the sheath 220 and the treatment tool channel 430. Therefore, when the sheath 220 is inserted through the treatment tool channel 430, the clip unit 1 can be easily rotated, and treatment using the clip unit 1 can be smoothly performed.

### Fifth Modification

### [Clip Device 300G]

Next, referring to FIG. 29, the clip device 300G according to a fifth modification will be described.

FIG. 29 is a side view showing the clip device 300G according to the fifth modification. The clip device 300G is a modified version of the clip device 300F. The clip device 300G differs from the clip device 300F in that it has a grip 200g.

The grip 200g is a member that is grasped by the user when the user rotates sheath 220 in circumferential direction S. The grip 200g is provided on sheath 220. The grip 200g is connected to the outer periphery of sheath 220. The grip 200g is a member that can slide on sheath 220 in longitudinal direction K. The outer diameter of the grip 200g is larger than the outer diameter of sheath 220. The grip 200g has a shape that is easy to grasp. The grip 200g has an outer diameter that is easy to grip.

The grip 200g is a member that has, for example, a slit formed on the inner diameter side, and is crushed when grasped by the user, allowing the user to the grip sheath 220 via the grip 200g. The grip 200g is, for example, a cylindrical rubber member. The user can move the grip 200g to a position that is easy to grasp and grasp it.

The provision of the grip 200g makes it easy for the user to rotate the sheath 220 in the circumferential direction S. This makes the clip device 300F easy to use.

The first embodiment of the present disclosure has been described above in detail with reference to the drawings, but the specific configuration is not limited to this embodiment and includes design changes and the like that do not depart from the gist of the present disclosure. In addition, the components shown in the above-mentioned embodiment and modified examples can be configured in appropriate combinations.

What is claimed is:

1. A medical device, comprising:
a sheath extending in a longitudinal direction;
a treatment portion disposed at a distal end of the sheath;
a wire inserted into the sheath and to which the treatment portion is fixed;
a handle disposed at a proximal end of the sheath and to which the wire is fixed; and
a transmission member disposed on an outer periphery of the sheath and extending from the handle to the distal end,
wherein the treatment portion, the wire, the handle, and the transmission member are rotatable in a circumferential direction with respect to the longitudinal direction relative to the sheath.

2. The medical device according to claim 1, wherein the transmission member is formed in a cylindrical shape through which the sheath is inserted.

3. The medical device according to claim 1, wherein
the handle includes
a handle body, and
a slider supported by the handle body and movable in the longitudinal direction,
the wire is connected to the slider and movable in the longitudinal direction in conjunction with the slider, and
the transmission member has a higher flexibility than the handle and is bendable along the sheath.

4. The medical device according to claim 1, wherein
an outer diameter of the handle is larger than an inner diameter of an endoscope channel through which the treatment portion can be inserted, and
an outer diameter of the transmission member is smaller than an inner diameter of the endoscope channel.

5. The medical device according to claim 1, wherein a length of the transmission member is longer than a length of the handle in the longitudinal direction.

6. The medical device according to claim 1, wherein a length of the transmission member is shorter than a length of the sheath in the longitudinal direction.

7. The medical device according to claim 1, wherein
a length from a distal end of the treatment portion to a distal end of the transmission member in the longitudinal direction is shorter than a length of an endoscope channel through which the treatment portion can be inserted.

8. The medical device according to claim 7, wherein the distal end of the transmission member is accommodated in the endoscope channel in a state in which the treatment portion is inserted into the endoscope channel and protruding from a distal end of the endoscope channel.

9. The medical device according to claim 1, wherein
a length from a distal end of the transmission member to a forceps port of an endoscope channel is 30 cm or less in a state in which the treatment portion is inserted into the endoscope channel and protruding from a distal end of the endoscope channel.

10. The medical device according to claim 1, wherein
the handle includes
a first part connected to the transmission member and rotatable in conjunction with the transmission member, and
a second part connected to be rotatable relative to the first part.

11. The medical device according to claim 10, wherein
the handle includes
a handle body, and
a slider supported by the handle body and movable in the longitudinal direction,
wherein the slider is disposed in the first part.

12. The medical device according to claim 10, wherein
the handle includes
a handle body,
a slider supported by the handle body and movable in the longitudinal direction,
wherein the slider is disposed in the second part.

13. The medical device according to claim 10, wherein
the handle includes
a stopper between the first part and the second part, configured to restrict a relative rotation between the first part and the second part.

14. The medical device according to claim 13, wherein the stopper is a friction member configured to generate friction between the first part and the stopper, and generate friction between the second part and the handle.

15. The medical device according to claim 14, wherein, in which the treatment portion is inserted into an endoscope channel through which the treatment portion can be inserted, a friction between the stopper and the first part and a friction between the stopper and the second part are determined by a rotational transmission property of the transmission member.

16. The medical device according to claim 13, wherein a friction between the stopper and a friction the first part and between the stopper and the second part are greater than 10 mN·m.

17. The medical device according to claim 1, wherein
the transmission member has a higher flexibility than the handle, and
the transmission member includes
an inner layer,
an outer layer, and
a middle layer that is disposed radially between the inner layer and the outer layer and has a harderness than the inner layer and the outer layer.

18. The medical device according to claim 1, wherein the transmission member can transition between a contracted state having a first length in the longitudinal direction and an extended state having a second length in the longitudinal direction that is longer than the first length.

19. A medical device, comprising:
a sheath extending in a longitudinal direction;
a treatment portion disposed at a distal end of the sheath;
a wire inserted through the sheath and to which the treatment portion is fixed; and
a handle disposed at a proximal end of the sheath and to which the wire is fixed,
wherein the sheath and the handle are fixed, and
the treatment portion, the wire, the handle, and the sheath are rotatable in a circumferential direction relative to a longitudinal direction in conjunction with each other.

20. The medical device according to claim 19, comprising
a grip connected to the sheath,
wherein the grip is slidable on the sheath and has an outer diameter larger than an outer diameter of the sheath.
